(19) United States
(12) Patent Application Publication
(10) Pub. No.: US 2006/0173481 A1
(43) Pub. Date: Aug. 3, 2006

(54) Ring stripper with tissue dissecting element

(75) Inventor: Ted S. Thorson, Sunnyvale, CA (US)

Correspondence Address: HAYNES BEFFEL & WOLFELD LLP, P O BOX 366, HALF MOON BAY, CA, 94019, US

(73) Assignee: VASCULAR ARCHITECTS, INC., a Delaware Corporation, San Jose, CA (US)

(21) Appl. No.: 11/050,406

(22) Filed: Feb. 3, 2005

(51) Int. Cl. A61B 17/00

(52) U.S. Cl. 606/190

(57) ABSTRACT

A ring stripper comprises a shaft and a tissue separator ring joined to the distal end of the shaft at a junction. A tissue dissecting element extends from the junction and has a leading surface with at least a portion of the leading surface being located distally of the ring. The tissue dissecting element may, and preferably does, extend along the shaft axis. The ring stripper may also include means for enhancing tissue dissection of the tissue dissecting element, such as by mechanically vibrating or reciprocating or otherwise moving at least the leading surface of the dissecting element or by heating the tissue to be dissected, or a combination thereof.

### FEDERALLY SPONSORED RESEARCH OR DEVELOPMENT

None.

### BACKGROUND OF THE INVENTION

This invention relates to a device or assembly for the treating of blood vessels and more specifically to a device or assembly for performing a remote endarterectomy or dilatation and to an endarterectomy method using the device.

It is known that narrowing or blockages (thromboses) can occur in blood vessels, particularly in older people. This is often caused by the effects of deposits on the inside walls of the blood vessels leading to hardening or calcifying of the blood vessels. This has dangerous consequences for the health, because the quantity of blood now able to flow through the blood vessel is drastically reduced. For effective blood circulation to occur, and to avoid possible limb amputation for example, any blockage or obstacle in the blood vessels must be removed.

One conventional method for treating hardening of the blood vessels involves a complicated operation. In the case of the artery between the groin and knee, this is quite a severe operation. The patient is cut open at the groin and the knee, whereafter the artery is completely removed and replaced by an artificial artery. This can be especially hard on and dangerous for older people, particularly because of the duration of the operation. The operation is also expensive and requires a lengthy hospital recovery period for the patient. Additionally, there is a danger of rejection of the artificial blood vessel by the body, which can lead to further post operation complications.

Another procedure for treating totally or partially blocked blood vessels, called endarterectomy, separates the inner layer of the blood vessel, the so called tunica-intima or intimal lining or intima, from the blood vessel wall using a ring stripper. Ring strippers are used to separate the intimal lining from a blood vessel. The intimal lining, which has been separated from the vessel wall over the length of the blockage, is then removed from the patient along with the blockage. A new intimal lining then grows back to replace the removed intimal lining. See, for example, U.S. Pat. Nos. 2,944,552 and 4,621,636.

Conventional endarterectomy procedures are typically carried out using two incisions, one on either side of the blockage. The first incision permits access to the vessel by the instrument; the second incision permits access to the distal end of the separated intima so the separated intima may be severed from the vessel wall and then removed through the first incision. U.S. Pat. No. 6,328,749 discloses a device eliminating the need for two incisions.

### BRIEF SUMMARY OF THE INVENTION

The present invention is directed to an improved ring stripper. One of the problems with conventional ring strippers is that they are somewhat flexible and therefore cannot overcome significant resistance to their passage between the intimal layers. If this occurs, the procedure is typically halted. This invention is an improvement over a conventional ring stripper, and includes a dissecting element which extends distally from the intersection of the shaft and the ring. The addition of the dissecting element allows the physician to continue with the dissection even when significant resistance to the passage of the ring between the intimal layers is encountered.

The ring stripper comprises a shaft with proximal and distal ends and a tissue separator ring joined to the distal end at a junction. The shaft has a longitudinally extending shaft axis. A tissue dissecting element extends from the junction. The dissecting element has a leading surface with at least a portion of the leading surface being located distally of the ring. The tissue dissecting element may, and preferably does, extend along the shaft axis. The ring stripper may also include means for enhancing tissue dissection of the tissue dissecting element, such as by mechanically vibrating or reciprocating or otherwise moving at least the leading surface of the dissecting element or by heating the tissue to be dissected, or a combination thereof.

Various features and advantages of the invention will appear from the following description in which the preferred embodiments have been set forth in detail in conjunction with the accompanying drawings.

### BRIEF DESCRIPTION OF THE DRAWINGS

FIG. 1 illustrates the distal portion of a conventional ring stripper;

FIG. 2 illustrates a ring stripper with dissecting element made according to the invention;

FIG. 3 is a side elevational view of the ring stripper of FIG. 2;

FIG. 4 is a view of an alternative embodiment similar to that of FIG. 3 in which the dissecting element has a curved distal end;

FIG. 5 is a cross-sectional view of the V-shaped leading surface an alternative embodiment of the dissecting element of FIG. 3;

FIG. 6 is a view of an alternative embodiment similar to that of FIG. 3 in which the dissecting element in the shaft are oriented generally perpendicular to the ring;

FIG. 7 is a plan view of the distal end of the dissecting element of FIG. 2 showing the straight leading surface; and

FIGS. 8-12 are views similar to that of FIG. 7 showing alternative embodiments of the distal end of the dissecting element in which the leading surfaces have different shapes;

FIG. 13 illustrates the distal end of a dissecting element having a leading surface that can be mechanically moved to enhance tissue dissection.

### DETAILED DESCRIPTION OF THE INVENTION

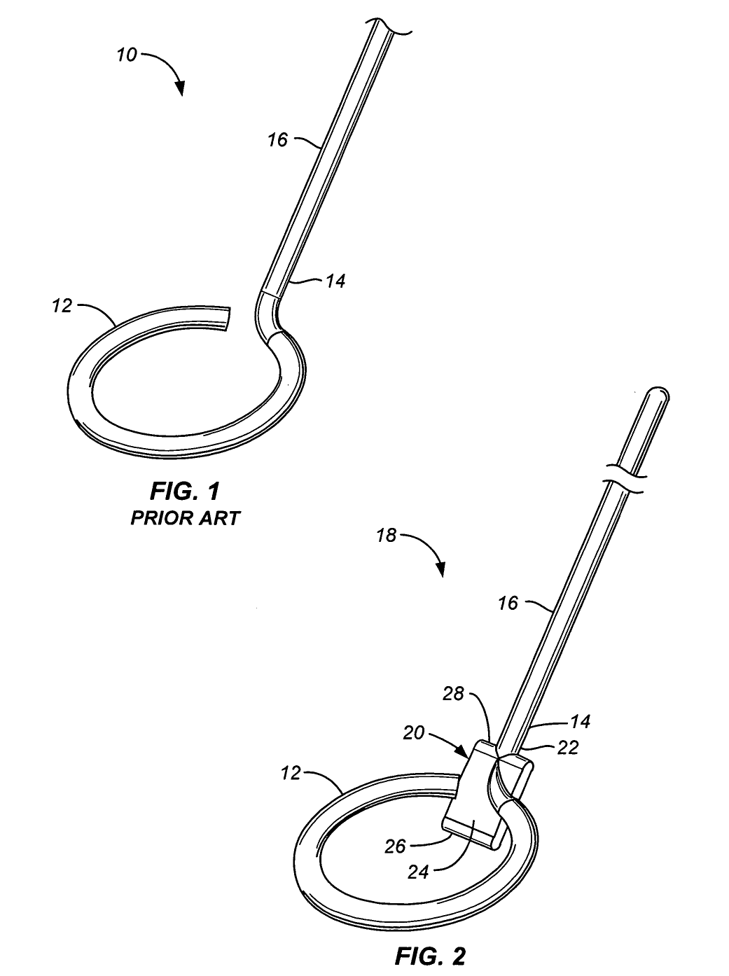

FIG. 1 illustrates the distal portion of a conventional ring stripper 10 comprising a ring 12 secured to the distal end 14 of a shaft 16. Ring strippers are used to separate the intimal lining from a blood vessel, typically because it is diseased. One of the problems with conventional ring strippers is that they are somewhat flexible and therefore cannot overcome significant resistance to their passage between the intimal layers. If this occurs, the procedure is typically halted.

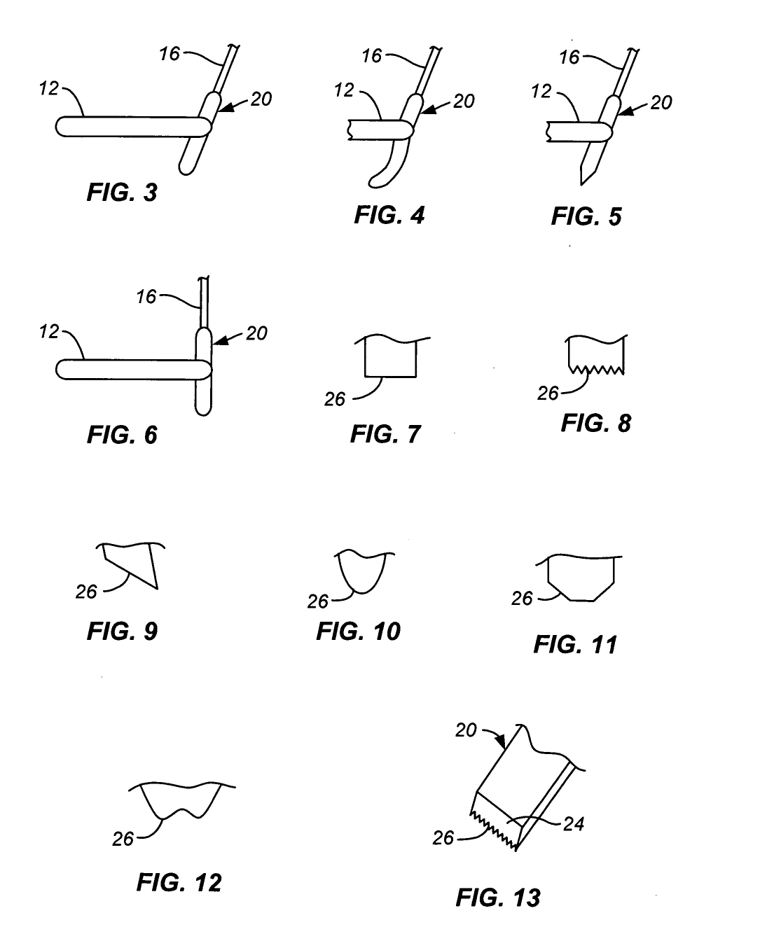

FIGS. 2 and 3 illustrate a ring stripper 18 made according to the invention similar to ring stripper 10 and with like reference numerals referring to like elements. Ring stripper 18 comprises a tissue dissecting element 20 extending from the junction 22 of the distal end 14 of shaft 16 and ring 12. Dissecting element 20 has a distal end 24 with a leading surface or edge 26 located distally of ring 12. Dissecting element 20 also has a trailing surface 28 located proximally of ring 12. The addition of dissecting element 20 allows the physician to continue with the dissection even when significant resistance to the passage of ring 12 between the intimal layers of a blood vessel is encountered. Leading surface 26, being aligned with shaft 16, is used to help overcome any localized or general significant resistance to the passage of ring 12.

Ring stripper 18 may be made of one or more conventional or unconventional materials, such as stainless steel, nickel-titanium alloys or polymers. Also, one or both of ring 12 and shaft 16 may be made of a different material from dissecting element 20 and leading surface or edge 26 of distal end 24 may be made of a material different from the remainder of dissecting element 20. All or part of ring stripper 18 may be coated with, for example, a suitable low friction material.

The shape of dissecting element 20 may be selected according to personal preferences of the physician as well as what is expected to be, and what is, encountered during the dissection procedure. FIG. 4 is a view of an alternative embodiment similar to that of FIG. 3 in which dissecting element 20 has a curved distal end 24. FIG. 5 is a cross-sectional view of a V-shaped leading surface 26A of an alternative embodiment of the dissecting element of FIG. 3. Such a leading surface may be useful when it is necessary to cut or otherwise sever an obstruction along the path of ring 12. FIG. 6 is a view of an alternative embodiment similar to that of FIG. 3 in which dissecting element 20 and shaft 16 are oriented generally perpendicular to the ring as opposed to at an angle in the FIG. 2 embodiment.

FIG. 7 is a plan view of distal end 24 of dissecting element 20 of FIG. 2 showing the straight leading surface 26. FIGS. 8-12 are views similar to that of FIG. 7 showing alternative embodiments of distal ends 24 and leading surfaces 26 of the dissecting element 20. FIGS. 8-12 show toothed, beveled, rounded, truncated triangular and waved or generally W-shaped leading surfaces 26, respectively.

In some situations in may be desired to enhance the tissue dissection ability of dissecting element 20. One way of doing so is illustrated in FIG. 13 in which distal end 24 of dissecting element 20 has a leading surface 26 that can be mechanically vibrated or reciprocated or otherwise moved to enhance tissue dissection. Another way of doing so would be to locally heat leading surface 26 with or without mechanically moving the leading surface.

Other modification and variation can be made to the disclosed embodiments without departing from the subject of the invention as defined in following claims.

Any and all patents, patent applications and printed publications referred to above are incorporated by reference.

What is claimed is:

1. A ring stripper of the type comprising a shaft with proximal and distal ends and a tissue separator ring joined to the distal end at a junction, the shaft having a shaft width at the distal end, the shaft having a longitudinally extending shaft axis at the distal end, the improvement comprising:
a tissue dissecting element extending from the junction; and
the dissecting element comprising a leading surface, at least a portion of the leading surface located distally of the ring.

2. A ring stripper according to claim 1, wherein the tissue dissecting element extends along the shaft axis.

3. A ring stripper according to claim 1, wherein the dissecting element has a length and a width and a thickness.

4. A ring stripper according to claim 3, wherein the width of the dissecting element is substantially greater than the shaft width.

5. A ring stripper according to claim 3, wherein the leading surface extends along a straight line relative to the width of the dissecting element.

6. A ring stripper according to claim 3, wherein at least a portion of the leading surface extends along a straight line relative to the width of the dissecting element.

7. A ring stripper according to claim 1, wherein the leading surface has a curved cross-sectional shape.

8. A ring stripper according to claim 1, wherein the leading surface has a V cross-sectional shape.

9. A ring stripper according to claim 1, wherein the leading surface has a saw tooth shape.

10. A ring stripper according to claim 1, further comprising means for enhancing tissue dissection by the tissue dissecting element.

11. A ring stripper according to claim 10, wherein the tissue dissection enhancing means comprises a mechanically movable leading surface of the dissecting element.

12. A ring stripper according to claim 10, wherein the tissue dissection enhancing means comprises means for heating the tissue to be dissected.

13. A ring stripper according to claim 1, wherein the tissue dissecting element is flat.

14. A ring stripper according to claim 1, wherein the tissue dissecting element comprises a trailing surface located proximally of the ring.
